(19) United States
(12) Patent Application Publication
(10) Pub. No.: US 2024/0423711 A1
(43) Pub. Date: Dec. 26, 2024

(54) LASER ABLATION DEVICE AND METHODS FOR USING THE SAME

(71) Applicant: Medtronic, Inc., Minneapolis, MN (US)

(72) Inventor: Janardan Vaidyanathan, Thane (IN)

(21) Appl. No.: 18/748,478

(22) Filed: Jun. 20, 2024

Related U.S. Application Data
(60) Provisional application No. 63/521,992, filed on Jun. 20, 2023.

(51) Int. Cl. A61B 18/20; A61B 18/00

(52) U.S. Cl. CPC A61B 18/201; A61B 2018/00023; A61B 2018/00577; A61B 2018/00642; A61B 2018/00702; A61B 2018/00714; A61B 2018/00744; A61B 2018/00791

(57) ABSTRACT

A system according to at least one embodiment of the present disclosure includes: an ablation device, including: a tubular housing extending from a proximal end to a distal end; a laser fiber disposed at least partially within the tubular housing, the laser fiber capable of generating ablation energy dissipated from the distal end of the tubular housing; an inflow channel disposed at least partially within the tubular housing, the inflow channel fluidically connectable to a coolant reservoir to dispense a coolant into the distal end of the tubular housing; and a plurality of outflow channels each disposed at least partially within the tubular housing and that enable removal of the coolant from the distal end of the tubular housing.

### CROSS-REFERENCE TO RELATED APPLICATIONS

This application claims the benefit of and priority to U.S. Provisional Patent Application Ser. No. 63/521,992 filed Jun. 20, 2023, the entire disclosure of which is incorporated by reference herein.

### TECHNICAL FIELD

The present disclosure is generally directed to surgeries and surgical procedures, and relates more particularly to surgical ablation.

### BACKGROUND

Various surgical tools are often required to successfully complete surgical procedures. Certain types of tools may be required for different procedures. Some surgical tools utilize heat to ablate anatomical tissues.

### BRIEF SUMMARY

Example aspects of the present disclosure include:

A system according to at least one embodiment of the present disclosure comprises: an ablation device, comprising: a housing extending from a proximal end to a distal end; a laser fiber disposed at least partially within the housing, wherein the laser fiber is configured to generate ablation energy dissipated from the distal end of the housing to ablate anatomical tissue; at least one inflow channel disposed at least partially within the housing, the at least one inflow channel fluidically connectable to a coolant reservoir to dispense a coolant into the distal end of the housing; and a plurality of outflow channels each disposed at least partially within the housing and that enable removal of the coolant from the distal end of the housing, wherein the at least one inflow channel and the plurality of outflow channels are positionable in different sections of the housing to control a heat profile associated with the ablation energy; a processor; and a memory storing data thereon that, when executed by the processor, enable the processor to: determine a desired orientation of the ablation device relative to the anatomical tissue; adjust an ablation setting associated with the ablation device to achieve the desired orientation; and cause the laser fiber to generate the ablation energy.

Any of the features herein, wherein the at least one inflow channel comprises two inflow channels.

Any of the features herein, wherein the plurality of outflow channels comprises three outflow channels.

Any of the features herein, wherein the coolant comprises saline, water, carbon dioxide (CO2) gas, or a combination thereof.

Any of the features herein, wherein the ablation setting comprises a temperature of the coolant, an inflow rate of the coolant, an outflow rate of the coolant, a type of coolant, or a combination thereof.

Any of the features herein, wherein the type of coolant is changed from a first coolant type to a second coolant type.

Any of the features herein, wherein a temperature of the second coolant type is different from a temperature of the first coolant type.

Any of the features herein, wherein the at least one inflow channel, at least one outflow channel of the plurality of outflow channels, or a combination thereof is at least partially coiled around a cannula containing the laser fiber.

Any of the features herein, wherein adjusting the ablation setting comprises changing an outflow rate of at least one outflow channel of the plurality of outflow channels.

Any of the features herein, wherein a diameter of the at least one inflow channel is different from a diameter of at least one outflow channel.

Any of the features herein, wherein the plurality of outflow channels comprises a first outflow channel and a second outflow channel, wherein the first outflow channel has a first outflow rate, and wherein the second outflow channel has a second outflow rate different than the first outflow rate.

Any of the features herein, wherein the processor causes the ablation energy to be generated at a first power at a first time, and at a second power greater than the first power at a second time later than the first time.

Any of the features herein, wherein the data, when processed by the processor, further enable the processor to: receive, when the ablation energy is generated at the first power, anatomical tissue temperature information associated with a temperature of the anatomical tissue; and adjust, based on the anatomical tissue temperature information and a surgical plan, the ablation setting associated with the ablation device.

Any of the features herein, wherein the different sections comprise a first section and a second section, wherein the at least one inflow channel is disposed in the first section, and wherein at least one outflow channel of the plurality of outflow channels is disposed in the second section.

Any of the features herein, wherein the different sections comprise a first section, a second section, and a third section, wherein the laser fiber and the at least one inflow channel are disposed in the first section, wherein a first outflow channel of the plurality of outflow channels is disposed in a second section, and wherein a second outflow channel of the plurality of outflow channels is disposed in the third section.

Any of the features herein, wherein the different sections comprise a first section, a second section, a third section, and a fourth section, wherein the laser fiber and the at least one inflow channel are disposed in the first section, wherein a first outflow channel of the plurality of outflow channels is disposed in the second section, wherein a second outflow channel of the plurality of outflow channels is disposed in the third section, and wherein a third outflow channel of the plurality of outflow channels is disposed in the fourth section.

A method according to at least one embodiment of the present disclosure comprises: determining a current orientation of an ablation device relative to anatomical tissue; determining a desired orientation of the ablation device relative to the anatomical tissue; adjusting an ablation setting associated with the ablation device to control a heat profile of ablation energy associated with the ablation device to achieve the desired orientation; and causing a laser fiber of the ablation device to generate the ablation energy.

Any of the features herein, further comprising: connecting the ablation device to a coolant reservoir; and setting an inflow rate of a coolant into the ablation device to match an outflow rate of the coolant from the ablation device.

Any of the features herein, wherein the coolant comprises saline, water, carbon dioxide (CO2) gas, or a combination thereof.

Any of the features herein, wherein adjusting the ablation setting comprises: changing an outflow rate of at least one outflow channel of a plurality of outflow channels associated with the ablation device.

Any of the features herein, wherein determining the current orientation of the ablation device comprises: causing the laser fiber to generate the ablation energy at a first power; and receiving anatomical tissue temperature information associated with a temperature of the anatomical tissue.

Any of the features herein, further comprising: adjusting, based on the anatomical tissue temperature information and a surgical plan, the ablation setting associated with the ablation device.

Any of the features herein, wherein the ablation energy at the first power is insufficient to ablate the anatomical tissue.

Any of the features herein, wherein the ablation setting comprises a temperature of a coolant, an inflow rate of the coolant, an outflow rate of the coolant, a type of coolant, or a combination thereof.

Any of the features herein, further comprising: changing the type of coolant from a first coolant type to a second coolant type.

Any of the features herein, wherein a temperature of the second coolant type is different from a temperature of the first coolant type.

A system according to at least one embodiment of the present disclosure comprises: an ablation device, comprising: a tubular housing extending from a proximal end to a distal end; a laser fiber disposed at least partially within the tubular housing, the laser fiber configured to generate ablation energy dissipated from the distal end of the tubular housing; an inflow channel disposed at least partially within the tubular housing, the inflow channel fluidically connectable to a coolant reservoir to dispense a coolant into the distal end of the tubular housing; and a plurality of outflow channels each disposed at least partially within the tubular housing and that enable removal of the coolant from the distal end of the tubular housing.

Any of the features herein, further comprising: a processor; and a memory storing data thereon that, when processed by the processor, enable the processor to: determine a desired orientation of the ablation device relative to the anatomical tissue; adjust an outflow rate associated with at least one outflow channel to change a heat profile associated with the ablation energy to achieve the desired orientation; and cause the laser fiber to generate the ablation energy.

A system according to at least one embodiment of the present disclosure comprises: a processor; and a memory storing data thereon that, when processed by the processor, enable the processor to: determine a desired orientation of an ablation probe relative to anatomical tissue; adjust an operation setting associated with at least one outflow channel of the ablation probe to achieve the desired orientation; and cause a laser fiber of the ablation probe to generate the ablation energy.

Any of the features herein, further comprising a coolant reservoir, wherein the ablation probe comprises: a housing extending from a proximal end to a distal end; an inflow channel disposed at least partially within the housing, the at least one inflow channel fluidically connectable to the coolant reservoir to dispense a coolant into the distal end of the housing; and a plurality of outflow channels each disposed at least partially within the housing and that enable removal of the coolant from the distal end of the housing, wherein the laser fiber is disposed at least partially within the housing, and wherein the ablation energy is dissipated from the distal end of the housing to ablate the anatomical tissue.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a perspective view of aspects of an ablation probe according to at least one embodiment of the present disclosure;

FIG. 1B is a side view of aspects of the ablation probe according to at least one embodiment of the present disclosure;

FIG. 1C is a cross section view of the ablation probe with coolant channels in a first configuration according to at least one embodiment of the present disclosure;

FIG. 1D is a cross section view of the ablation probe with coolant channels in a second configuration according to at least one embodiment of the present disclosure;

FIG. 1E is a cross section view of the ablation probe with coolant channels in a third configuration according to at least one embodiment of the present disclosure;

FIG. 2 is a block diagram of a system according to at least one embodiment of the present disclosure;

FIG. 3 is a flowchart according to at least one embodiment of the present disclosure; and

FIG. 4 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Current ablation systems can contain a radial diffusion fiber. The radial diffusion fiber enables volumetric ablation in a concentric hyperthermia zone (assuming normal tissue is symmetrically distanced from the laser diffusion tip). Skewed tissue ablation around the laser diffusion fiber can be challenging using these ablation systems, since a torque effect associated with rotating a concentric catheter results in some amount of under or over steer. While pull back methods (where the radial diffusion fiber is slowly retracted from the catheter to ablate anatomical tissue along the trajectory of the inserted catheter) can be used, such methods result in linear ablation but do not enable annular ablation. With current ablation systems, the treating physician (e.g., a neurosurgeon) chooses between either a radial laser diffusion fiber or a directional laser diffusion fiber at the beginning of the procedure. However, the directional laser window requires manual or mechanical adjustment in order to reorient or steer the treatment beam. It is desirable to provide a directionally non-uniform dispersion laser diffusion fiber whose treatment beam (whether a radial beam or a linear beam) can be programmed during delivery of the treatment.

According to at least one embodiment of the present disclosure, an ablation system can provide a multi-directional radial diffusion fiber. The field of diffusion can be adjusted by augmenting the flow rates of the coolant used in the ablation device to ablate the desired anatomical tissue. For example, flow rates or coolant types and in-flow temperatures can be configured during treatment, and the orientation of the ablation device relative to the anatomical tissue can be verified, before the therapy is delivered. As a result, the ablation device enables contoured ablation of anatomical tissue while preserving adjacent structures.

According to at least one embodiment of the present disclosure, non-uniform cooling of the laser fiber tip eliminates the need for the physician to commit to and be limited by either a radial or side diffusion method before commencing the treatment. Due to the augmented flow rates of the coolant, there are areas of the laser fiber tip where there is greater cooling, which acts as a heat sink to absorb a greater amount of heat, resulting in less heat transmitted to the anatomical tissue proximate the ablation device. In some embodiments, the cannula lumen may be partitioned into sections, and different coolants (e.g., gases and/or liquids) may be used in the different partitions.

According to at least one embodiment of the present disclosure, an ablation surgical procedure may comprise connecting an inflow port (also referred to herein as an inflow channel) to a cooling fluid (e.g., a pressured gas or liquid). An outflow port (also referred to herein as an outflow channel) may then be selected to induce a temperature gradient by increasing the suction applied to the outflow port. In some examples, the inflow ports and the outflow ports may be reassigned or switchable, such that any given port could operate as an inflow port or an outflow port. For example, a first port could be used as an inflow port by connecting the first port to a coolant source (e.g., a coolant reservoir) or alternatively could be used as an outflow port by connecting the first port to a suction mechanism designed to extract the coolant from the ablation device. Additionally or alternatively, there may be differential suction applied to a plurality of outflow ports, such as when a single outflow port is disposed in a first section and two outflow ports are disposed in a second, different section. In this example, the suction may be applied to all three outflow ports, but the existence of the two outflow ports may result in the second section experiencing greater suction, inducing a temperature gradient. The inflow rate may then be set to match the total set suction outflow rate, such that the amount of coolant in the ablation device remains stable. In some embodiments, the outflow port and/or the inflow port may be coiled around the longitudinal axis of the laser fiber. A test laser beam may then be shone on the anatomical tissue; in other words, the laser fiber may be caused to generate ablation energy that is transferred into the anatomical tissue. The test laser beam may be generated at a power that falls below the threshold power required to ablate the anatomical tissue. As a result, the anatomical tissue may be heated without ablation. A temperature map of the anatomical tissue may be generated (e.g., based on thermal imaging, based on measurements from one or more temperature sensors, etc.) to identify the heat profile of the anatomical tissue. For example, if the outflow rate is set to be higher on a first side of the ablation device than on a second side of the ablation device, the first side of the ablation device may operate as a heat sink, and the anatomical tissue on the first side of the ablation device may receive less heat. In this example, the second side of the ablation device may receive a greater amount of heat, and will heat up to a higher temperature than the anatomical tissue on the first side of the ablation device. Based on the heating of the anatomical tissue, a reference orientation of the ablation device may be determined relative to the anatomical tissue.

Once the reference orientation has been determined, the treatment direction orientation of the treatment beam may be determined. The direction may be determined based on a surgical plan that specifies the location of patient anatomy and a tumor. The direction may then be compared to the orientation reference marker to determine the location of the patient anatomy and the tumor relative to the ablation device. Based on the position of the patient anatomy and the tumor, the inflow and outflow rates of the coolant in the ablation device may be set to tailor the predicted damage model to the patient. In other words, the inflow and outflow rates may be set such that the tumor will receive the ablation energy. Then, and as discussed above, the ablation device may generate a test laser beam and generate a temperature map of the patient anatomy and the tumor to confirm that the settings enable the ablation device to ablate the tumor. The treatment may then be performed, and the results may be confirmed by capturing one or more medical images of the patient (e.g., an MRI scan).

Additionally or alternatively, one or more other settings of the ablation device may be adjusted to change the heat profile of the anatomical tissue. For example, the temperature of the inflow coolant may be adjusted. The inflow and outflow tubing diameter may be effectively adjusted by offsetting the laser fiber within the cannula toward one side of the cannula. Different partitions of the ablation device may have different coolants (e.g., a first partition has a gas coolant while a second partition has a liquid coolant) with different propensities to absorb heat, resulting in a temperature gradient.

Embodiments of the present disclosure provide technical solutions to one or more of the problems of (1) physicians choosing and being limited by a single ablation technique for a surgery or surgical procedure, and (2) drawbacks associated with directional ablation techniques.

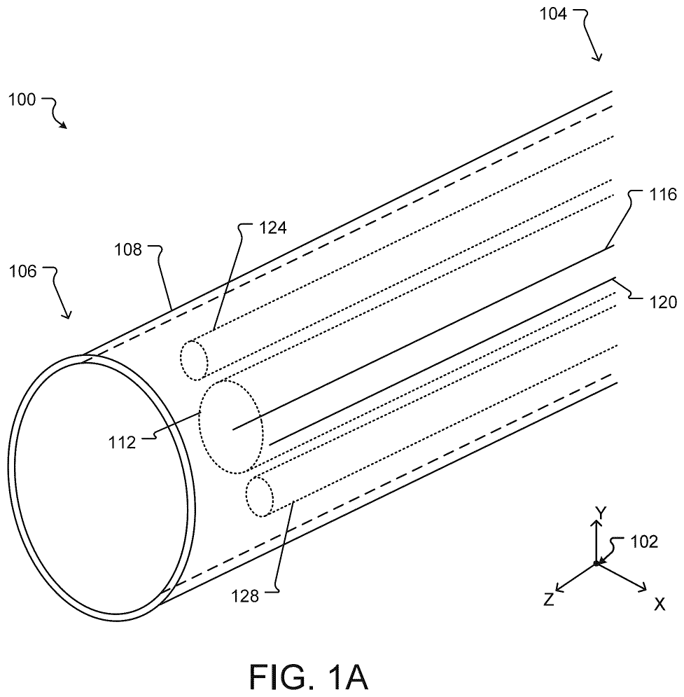

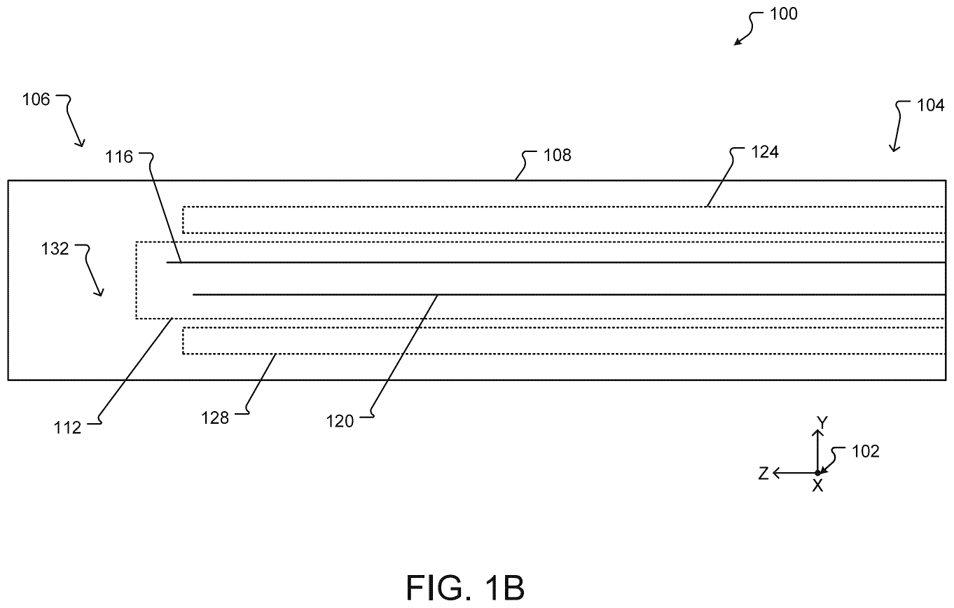

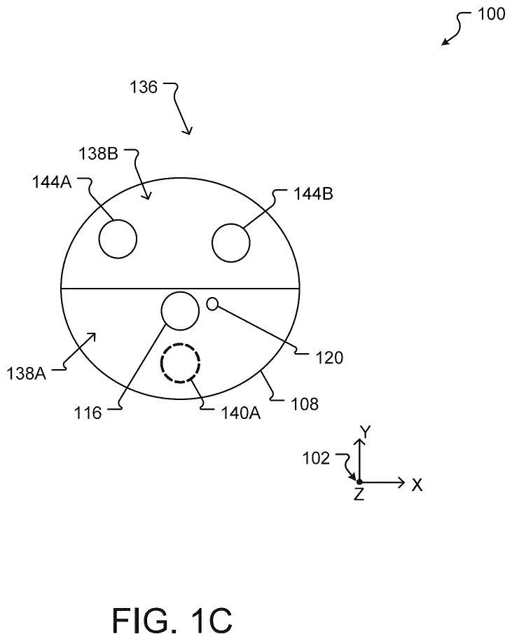

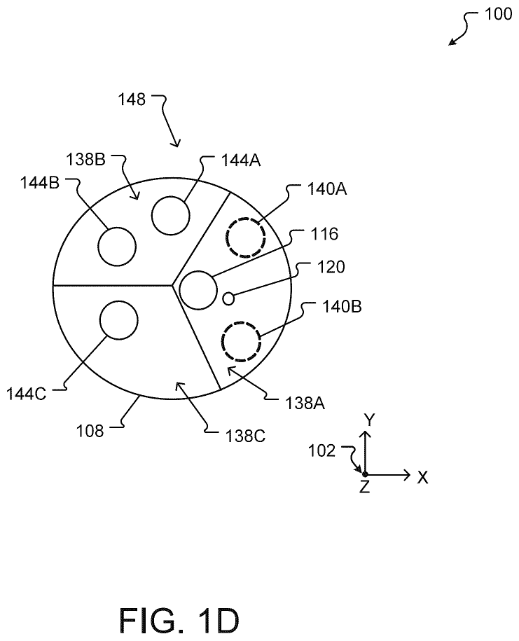

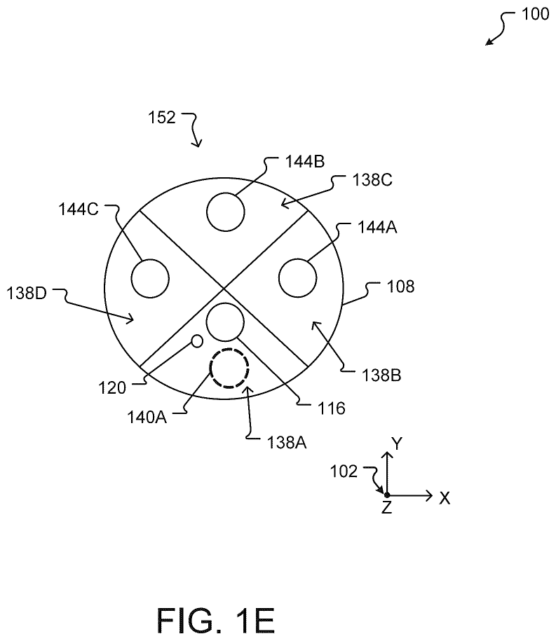

Turning first to FIGS. 1A-1E, aspects of an ablation probe 100 according to at least one embodiment of the present disclosure are shown. The ablation probe 100 may be used to perform ablation of anatomical tissue and/or to carry out one or more other aspects of one or more of the methods discussed herein. The ablation probe 100 includes a housing 108 that extends from a proximal end 104 to a distal end 106. In some embodiments, the proximal end 104 extends outside of the patient, such that a first portion of the ablation probe 100 closer to the distal end 106 is implanted in the patient while a second portion of the ablation probe 100 closer to the proximal end 104 extends outside of the patient when the ablation probe 100 is inserted into the patient. The housing 108 comprises a center cannula 112 that includes a laser fiber 116 and a temperature monitor 120, an inflow channel 124, and an outflow channel 128. In some embodiments, the housing 108 may be tubular in shape, such that the housing 108 can linearly slide into the surgical site when the ablation probe 100 is inserted into the patient.

In some embodiments, reference may be made to dimensions, angles, directions, relative positions, and/or movements associated with one or more components of the ablation probe 100 with respect to a coordinate system 102. The coordinate system 102, as shown in the accompanying figures, includes three-dimensions comprising an X-axis, a Y-axis, and a Z-axis. Additionally or alternatively, the coordinate system 102 may be used to define planes (e.g., the XY-plane, the XZ-plane, and the YZ-plane) of the ablation probe 100. These planes may be disposed orthogonal, or at 90 degrees, to one another. While the origin of the coordinate system 102 may be placed at any point on or near the ablation probe 100, for the purposes of description, the axes of the coordinate system 102 are disposed along the same directions from figure to figure. Additionally or alternatively, the directionality of the X-axis, Y-axis, and Z-axis may be flipped, as noted with negative directionality (e.g., the negative X-axis direction is the opposite direction of the X-axis direction illustrated by the direction of the associated arrow). In some cases, the coordinate system 102 may be defined based on or using cartesian coordinates, cylindrical coordinates, polar coordinates, combinations thereof, and/or the like.

The laser fiber 116 conducts electrical current (e.g., an RF current) therethrough to generate ablation energy (e.g., heat) to ablate anatomical tissue proximate the ablation probe 100. The laser fiber 116 may comprise an active electrode and a return electrode through which the current passes. The movement of current through the laser fiber 116 generates heat that is dissipated from the distal end 106 of the housing 108. In some embodiments, the laser fiber 116 may be attached to the generator 212 discussed below, which generates current. The generator 212 may be adjustable to vary the power or intensity of the heat generated by the laser fiber 116. The amount of heat generated by the laser fiber 116 may in some cases be adjusted based on the type or quality of the anatomical tissue to be ablated. In some embodiments, the laser fiber 116 may be moved proximally and/or distally in the center cannula 112. In some embodiments, the laser fiber 116 may be centered in the center cannula 112, while in other embodiments the laser fiber 116 may be disposed or moved toward the inner sidewall of the center cannula 112, such that the laser fiber 116 is off center from the center of the center cannula 112.

The temperature monitor 120 may be or comprise one or more sensors or thermally sensitive material capable of generating information that can be used to determine the temperature of the laser fiber 116 and/or the area surrounding the laser fiber 116. The temperature monitor 120 may generate one or more measurements that represent the temperature of the center cannula 112, the laser fiber 116, or the temperature of other components proximate the temperature monitor 120. In some embodiments, the temperature monitor 120 may extend into the housing 108 from the proximal end 104 of the ablation probe 100 and be disposed in the center cannula 112 of the ablation probe 100 to record data indicative of the temperature of the laser fiber 116. In some embodiments, the sensors and/or material composition of the temperature monitor 120 may be selected or chosen based on the type of surgery or surgical procedure, the anticipated temperature of the laser fiber 116, the anticipated amount of time needed to ablate anatomical tissue, the surgical plan, combinations thereof, and the like. In some embodiments, the ablation probe 100 may comprise multiple temperature monitors 120 to measure and/or monitor the temperature of one or more components of the ablation probe 100. In such embodiments, the multiple temperature monitors 120 may be used to determine the heating profile of the ablation probe 100, as discussed in further detail below. For example, measurements from the multiple temperature monitors 120 may be used to check the temperature gradient created by the ablation probe 100, which temperature gradient can be compared to a measured temperature map when a test beam is delivered. This may beneficially enable the user or the system controlling the ablation probe 100 to confirm that the heat generated by the ablation probe 100 is being absorbed by the desired anatomical tissue. In other words, the temperature monitors may provide additional measurements that can be used to verify that the ablation probe 100 is heating surrounding anatomical tissue in a desired manner.

The inflow channel 124 comprises a hollow tube that carries fresh coolant (e.g., water, saline, carbon dioxide (CO₂) gas, etc.) into the ablation probe 100 to cool (e.g., absorb heat from) the ablation probe 100. The outflow channel 128 comprises a hollow tube that transfers spent coolant that has absorbed heat while in the distal end 106 of the ablation probe 100 out of the ablation probe 100. The inflow channel 124 and the outflow channel 128 extend from the proximal end 104 and into the distal end 106 of the ablation probe 100. In some embodiments, the inflow channel 124 and/or the outflow channel 128 may be coiled or wrapped wholly or partially around the center cannula 112, such that a portion or the entirely of a longitudinal length of the center cannula 112 is cooled. In other words, the inflow channel 124 and/or the outflow channel 128 may be wrapped helically around a length of the center cannula 112 in the Z-axis direction of the coordinate system 102. In some cases, the center cannula 112 may be uniformly cooled, such that the anatomical tissue along and/or adjacent to the trajectory of the inserted catheter are preserved (e.g., do not experience sufficient heating to cause ablation).

The coolant is pumped into the distal end 106 through the inflow channel 124 to cool the ablation probe 100 while the laser fiber 116 generates ablation energy (e.g., heat), such as during an ablation procedure. In some embodiments, the housing 108 may comprise the center cannula 112 and an outer channel surrounding the center cannula 112. The outer channel may comprise the area occupied by the inflow channel 124, the outflow channel 128, and an interior cavity 132 of the distal end 106. The coolant may be dispensed from the inflow channel 124 and into the interior cavity 132 of the distal end 106 to absorb heat from the laser fiber 116 and/or one or more other components of the ablation probe 100 (e.g., an interior surface of the housing 108). The coolant may then flow out of the outer channel of the housing 108 through the outflow channel 128. In some embodiments, the outflow channel 128 may be connected to a pump that generates a negative pressure to carry the coolant out of the ablation probe 100. In some cases, the coolant flowing through the outflow channel 128 may be recirculated through the ablation probe 100. For example, the outflow channel 128 may extract the coolant from the ablation probe 100 and pump the coolant into a coolant container connected to the inflow channel 124, such that the coolant extracted by the outflow channel 128 is then again dispensed into the ablation probe 100 by the inflow channel 124. Additionally or alternatively, the coolant may be disposed of after being extracted or after having been used for a predetermined amount of time.

In some examples, the volume of the coolant flowing out of the ablation probe 100 through the outflow channel 128 may be monitored. Based on the volume of the coolant, a processor such as a processor 204 discussed below may determine that the coolant flow has been blocked or otherwise obstructed. In some cases and as a result of determining that coolant is not properly flowing, the processor may automatically disable one or more aspects of the ablation probe 100. For example, the processor may disable current flow through the laser fiber 116, such that the ablation probe 100 no longer generates ablation energy. This may be done, for example, to prevent inadvertent ablation along the path or trajectory of the inserted catheter.

The ablation probe 100 may, in some cases, include a plurality of inflow channels and a plurality of outflow channels, enabling a heat profile (e.g., a direction of greatest heat dissipation/temperature increase) associated with the ablation energy of the ablation probe 100 and/or associated with the heating of the surrounding anatomical tissue to be adjusted. In some embodiments, the plurality of inflow channels and the plurality of outflow channels may be positioned into different sections of the ablation probe 100, such that changes in flow rates of the plurality of inflow channels and/or changes in flow rates of the outflow channels create non-uniform cooling of the ablation probe 100 (or, in other words, temperature gradients), enabling a user or system to adjust the heat profile to effectively steer the heat to ablate anatomical tissue in different directions.

FIG. 1C depicts a cross section view of the ablation probe 100 with the inflow and outflow channels in a first configuration 136 according to at least one embodiment of the present disclosure. The first configuration 136 comprises the laser fiber 116, the temperature monitor 120, a first inflow channel 140A, a first outflow channel 144A, and a second outflow channel 144B. The first inflow channel 140A may be any inflow channel discussed herein (e.g., the inflow channel 124) or any similar inflow channel. The first outflow channel 144A and the second outflow channel 144B may each be any outflow channel discussed herein (e.g., the outflow channel 128) or any similar outflow channel.

The first configuration 136 partitions the housing 108 into a first section 138A and a second section 138B. In some embodiments, the partition between the first section 138A and the second section 138B may be a virtual partition created by differential flow rates of coolant from the inflow channel 124 and out through the first outflow channel 144A and the second outflow channel 144B. For example, the first outflow channel 144A may have a greater outflow rate than the second outflow channel 144B. As a result, the side of the ablation probe 100 containing first outflow channel 144A may have a greater heat sink effect, and dissipate less heat to the surrounding anatomical tissue than the side of the ablation probe 100 containing the second outflow channel 144B. As another example, the first outflow channel 144A and the second outflow channel 144B may have the same outflow rate. In this example, the first section 138A may have a greater heat sink effect (e.g., due to the lower temperature of the coolant dispensed from the inflow channel 124) than the second section 138B, resulting in greater heat dissipation from the second section 138B than from the first section 138A.

FIG. 1D depicts a cross section view of the ablation probe 100 with the inflow and outflow channels in a second configuration 148 according to at least one embodiment of the present disclosure. The second configuration 148 comprises the laser fiber 116, the temperature monitor 120, the first inflow channel 140A, a second inflow channel 140B, the first outflow channel 144A, the second outflow channel 144B, and a third outflow channel 144C. The second inflow channel 140B may be any inflow channel discussed herein (e.g., the inflow channel 124) or any similar inflow channel. The third outflow channel 144C be any outflow channel discussed herein (e.g., the outflow channel 128) or any similar outflow channel.

The second configuration 148 partitions the housing 108 into the first section 138A, the second section 138B, and a third section 138C. The first section 138A contains the laser fiber 116, the temperature monitor 120, the first inflow channel 140A, and the second inflow channel 140B; the second section 138B contains the first outflow channel 144A and the second outflow channel 144B; and the third section 138C contains the third outflow channel 144C.

While in the second configuration 148, the inflow rate of the first inflow channel 140A and/or the second inflow channel 140B; the outflow rate of the first outflow channel 144A, the second outflow channel 144B, and/or the third outflow channel 144C; the type of coolant dispensed into the ablation probe 100 by the first inflow channel 140A and/or the second inflow channel 140B; the temperature of the coolant dispensed into the ablation probe 100 by the first inflow channel 140A and/or the second inflow channel 140B; combinations thereof; and the like may be adjusted (e.g., based on user input, by a processor controlling pumps attached to the inflow channels and/or the outflow channels, etc.) to change the heating profile of the ablation probe 100 and/or the heating profile of the anatomical tissue proximate the ablation probe 100.

For example, the first inflow channel 140A may dispense a gas (e.g., CO₂ gas) into the ablation probe 100, and the first outflow channel 144A and the second outflow channel 144B may remove the gas from the ablation probe 100. The second inflow channel 140B may dispense a coolant different from the gas, such as water, saline, or another fluid, and the third outflow channel 144C may remove the other coolant from the ablation probe 100. Due to the different properties of the gas and the other coolant, the gas may absorb more heat than the other coolant (or vice versa). As a result, a temperature gradient may be created between the first section 138A and the second section 138B, between the first section 138A and the third section 138C, and/or between the second section 138B and the third section 138C.

Additionally or alternatively, the outflow rates of the first outflow channel 144A, the second outflow channel 144B, and the third outflow channel 144C may be set at the same value. In this example, the positioning of the first outflow channel 144A and the second outflow channel 144B in the second section 138B may result in a greater overall outflow rate of coolant in the second section 138B than in the third section 138C, which has the third outflow channel 144C. The difference in outflow rate may create a temperature differential between the second section 138B and the third section 138C, such that the third section 138C remains hotter and dissipates more heat to adjacent anatomical tissue than the second section 138B.

FIG. 1E depicts a cross section view of the ablation probe 100 with the inflow and outflow channels in a third configuration 152 according to at least one embodiment of the present disclosure. The third configuration 152 comprises the laser fiber 116, the temperature monitor 120, the first inflow channel 140A, the first outflow channel 144A, the second outflow channel 144B, and the third outflow channel 144C. The third configuration 152 partitions the housing 108 into the first section 138A, the second section 138B, the third section 138C, and a fourth section 138D. The first section 138A contains the laser fiber 116, the temperature monitor 120, and the first inflow channel 140A; the second section 138B contains the first outflow channel 144A; the third section 138C contains the second outflow channel 144B, and the fourth section 138D contains the third outflow channel 144C.

While in the third configuration 152, the inflow rate of the first inflow channel 140A; the outflow rate of the first outflow channel 144A, the second outflow channel 144B, and/or the third outflow channel 144C; the type of coolant and/or the temperature thereof dispensed into the ablation probe 100 by the first inflow channel 140A; combinations thereof; and the like may be adjusted (e.g., based on user input, by a processor controlling pumps attached to the inflow channels and/or the outflow channels, etc.) to change the heating profile of the ablation probe 100 and/or the heating profile of the anatomical tissue proximate the ablation probe 100.

For example, the outflow rate of the first outflow channel 144A may be set higher than the second outflow channel 144B and the third outflow channel 144C, and the second outflow channel 144B may be set higher than the third outflow channel 144C. In this example, the fourth section 138D may be hotter than the third section 138C and the second section 138B, creating a temperature gradient and resulting in anatomical tissue proximate the fourth section 138D receiving more heat than the anatomical tissue proximate the second section 138B or the third section 138C.

As another example, the second outflow channel 144B may be set at a higher outflow rate than either of the outflow rates of the first outflow channel 144A and the third outflow channel 144C. In this example, the third section 138C may be cooler and dissipate less heat to proximate anatomical tissue than the second section 138B and the fourth section 138D. Alternatively, the second outflow channel 144B may be set at a lower outflow rate than both the outflow rates of the first outflow channel 144A and the third outflow channel 144C. As a result, the third section 138C may be hotter and dissipate a greater amount of heat to proximate anatomical tissue than the second section 138B and the fourth section 138D.

In some example, the type of coolant used in the ablation probe 100 may be changed from a first coolant type to a second coolant type (e.g., from gas to liquid, from water to saline, from water to CO₂ gas, etc.) to change the heat dissipation of the ablation probe 100. The inflow temperature and/or the propensity of the coolant to absorb heat may be different for the first coolant type than for the second coolant type, resulting in different heat dissipations of the ablation probe 100. The change in coolant may occur, for example, when additional heat is required to ablate anatomical tissue. In such examples, the second coolant type may have less ability to absorb heat (whether due to a different specific heat, a higher starting temperature, combinations thereof, and/or the like) than the first coolant type. As a result, the current passing through the laser fiber 116 may remain the same while allowing the ablation probe 100 to dissipate additional heat to reach the ablation threshold. In some cases, the coolant type may be changed by a processor (e.g., the processor 204 discussed below). For example, the processor may disable one or more inflow channels associated with the first coolant type and enable one or more inflow channels associated with the second coolant type, such that the second coolant type flows through the ablation probe 100.

In yet another example, one or more of the first outflow channel 144A, the second outflow channel 144B, and/or the third outflow channel 144C may be disabled. For example, when the anatomical tissue to be ablated, such as a tumor, is proximate the third section 138C, the outflow rate of the second outflow channel 144B may be set to zero (e.g., a pump connected to the second outflow channel 144B is turned off). In such examples, the outflow of coolant through the first outflow channel 144A and the third outflow channel 144C may respectively create heat sinks in the second section 138B and the fourth section 138D, while the lack of outflowing coolant through the second outflow channel 144B may cause the third section 138C to dissipate heat. In this example, the third section 138C may emit heat to ablate the anatomical tissue proximate thereto, while anatomical tissue proximate the second section 138B and the fourth section 138D may be heated, but not ablated.

In some embodiments, the diameters of any one or more of the inflow channels may be different from the diameter of any other inflow channel. Similarly, the diameters of any one or more of the outflow channels may be different from the diameter of any other outflow channel. In some embodiments, the effective diameter of the outflow channel may be adjustable by positioning the laser fiber 116 offset from the center of the center cannula 112, such that the source of heat is moved relative to the outflow channel. For example, in the third configuration 152 the laser fiber 116 may be offset from the center cannula 112, such that the laser fiber 116 is positioned further along the X-axis of the coordinate system 102. As a result, coolant flowing through the first outflow channel 144A may absorb more heat per unit time than the third outflow channel 144C. The effective diameter of the third outflow channel 144C can then be considered as having changed, since the amount of total heat removed by the third outflow channel 144C has decreased.

In some embodiments, any of the partitions discussed herein may comprise actual partitions, such as walls running along the radial direction of the ablation probe 100 to physically isolate the sections 138A-138D from one another. Additionally or alternatively, the temperature gradient may be created by the use of different coolant types (e.g., gas coolant flowing through one inflow channel and liquid coolant flowing through another inflow channel). Furthermore, it is to be understood that while the first configuration 136, the second configuration 148, and the third configuration 152 are discussed herein, such configurations are in no way limiting, and additional or alternative configurations of the laser fiber 116, the temperature monitor 120, the inflow channels, and the outflow channels are possible.

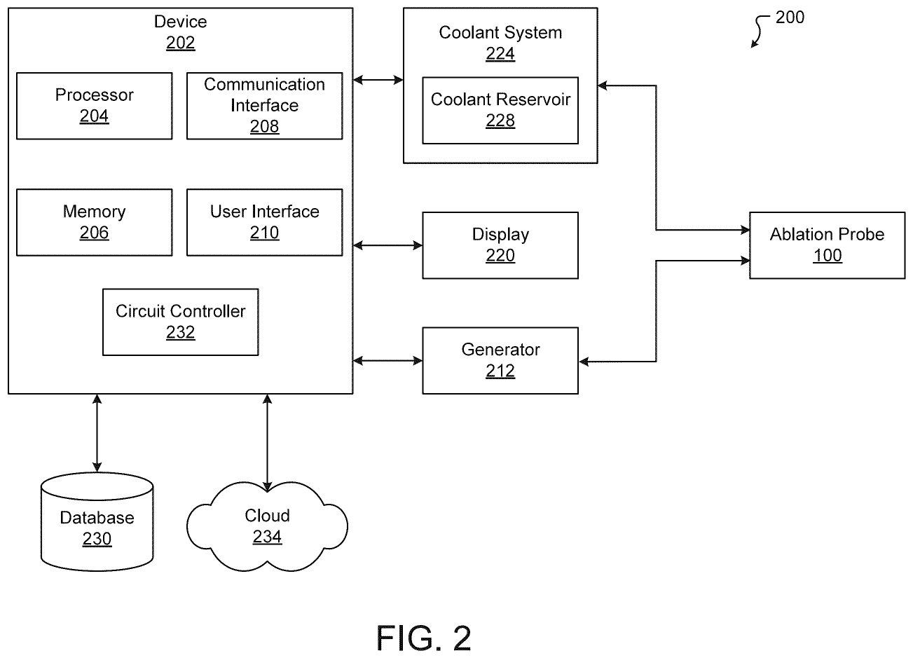

FIG. 2 depicts a block diagram of a system 200 according to at least one embodiment of the present disclosure. The system 200 may be used to control ablation of anatomical tissue using one or more ablation probes, enable user interaction and control of the one or more ablation probes, and/or to carry out one or more aspects of one or more of the methods disclosed herein. The system 200 comprises the ablation probe 100, a device 202, a generator 212, a display 220, a coolant system 224, a database 230, and a cloud or other network 234. In some embodiments, the system 200 may comprise additional or alternative components to those depicted in FIG. 2.

The device 202 comprises a processor 204, a memory 206, a communication interface 208, a user interface 210, and a circuit controller 232. In some embodiments, the device 202 may comprise more or fewer components than those depicted in FIG. 2.

The processor 204 of the device 202 may be any processor described herein or any similar processor. The processor 204 may be configured to execute instructions stored in the memory 206, which instructions may cause the processor 204 to carry out one or more computing steps utilizing or based on data received from the ablation probe 100, one or more components of the device 202, the generator 212, the display 220, the coolant system 224, the database 230, and/or the cloud 234.

The memory 206 may be or comprise RAM, DRAM, SDRAM, other solid-state memory, any memory described herein, or any other tangible, non-transitory memory for storing computer-readable data and/or instructions. The memory 206 may store information or data useful for completing, for example, any step of the methods described herein, or of any other methods. The memory 206 may store, for example, instructions that support one or more functions of the ablation probe 100. For instance, the memory 206 may store content (e.g., instructions) that, when executed by the processor 204, enable cooling of ablation probe 100 and/or ablation with the ablation probe 100. Such content, if provided as in instruction, may, in some embodiments, be organized into one or more applications, modules, packages, layers, or engines. Alternatively or additionally, the memory 206 may store other types of content or data that can be processed by the processor 204 to carry out the various method and features described herein. Thus, although various contents of memory 206 may be described as instructions, it should be appreciated that functionality described herein can be achieved through use of instructions, algorithms, data, and/or the like. The data, algorithms, and/or instructions may cause the processor 204 to manipulate data stored in the memory 206 and/or received from or via the ablation probe 100, one or more components of the device 202, the generator 212, the display 220, the coolant system 224, the database 230, and/or the cloud 234.

The communication interface 208 may be used for receiving data or other information from an external source (such as the generator 212, the display 220, the coolant system 224, the database 230, the cloud 234, and/or any other system or component not part of the system 200), and/or for transmitting instructions or other information to an external system or device (e.g., another device 202, the generator 212, the display 220, the coolant system 224, the database 230, the cloud 234, and/or any other system or component not part of the system 200). The communication interface 208 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 208 may be useful for enabling the device 202 to communicate with one or more other processors 204 or devices 202, whether to reduce the time needed to accomplish a computing-intensive task or for any other reason.

The user interface 210 may be or comprise a keyboard, mouse, trackball, monitor, television, screen, touchscreen, and/or any other device for receiving information from a user and/or for providing information to a user. The user interface 210 may be used, for example, to receive a user selection or other user input regarding any step of any method described herein. Notwithstanding the foregoing, any required input for any step of any method described herein may be generated automatically by the system 200 (e.g., by the processor 204 or another component of the system 200) or received by the system 200 from a source external to the system 200. In some embodiments, the user interface 210 may be useful to allow a surgeon or other user to modify instructions to be executed by the processor 204 according to one or more embodiments of the present disclosure, and/or to modify or adjust a setting of other information displayed on the user interface 210 or corresponding thereto.

Although the user interface 210 is shown as part of the device 202, in some embodiments, the device 202 may utilize a user interface 210 that is housed separately from one or more remaining components of the device 202. For example, the user interface 210 (or more generally the device 202) may be disposed within the display 220. In some embodiments, the user interface 210 may be located proximate one or more other components of the device 202, while in other embodiments, the user interface 210 may be located remotely from one or more other components of the device 202.

The generator 212 comprises one or more electrical components (e.g., batteries, resistors, capacitors, inductors, etc.) that facilitate the generation or modulation of current carried to the ablation probe 100. The circuit controller 232 controls the generator 212 to adjust the current (e.g., RF current) carried to the ablation probe 100. For example, the circuit controller 232 can control the generator 212 to generate a fixed current that is passed through the ablation probe 100. Additionally or alternatively, the circuit controller 232 can control the generator 212 to generate an alternating current. In some embodiments, the circuit controller 232 can control the generator 212 based on inputs from the user, instructions stored in the database 230, or the like. For example, the user may desire a first current, and may provide an input through the user interface 210 to the circuit controller 232 that instructs the generator 212 to generate the first current. The circuit controller 232 may then cause the generator 212 to generate the first current (e.g., by executing instructions stored in the memory 206).

The display 220 may be or comprise a screen or touchscreen that renders information related to the ablation for the user to view. In some embodiments, the user may be able to control one or more components of the system 200 through the display 220 (e.g., the display 220 may comprise the user interface 210). In one embodiment, the display 220 and the generator 212 may be disposed in the same housing. The type of information rendered to the display 220 is in no way limited, and some examples of information related to the surgery include an amount of power supplied to the ablation probe 100; information about a temperature of the ablation probe 100 (e.g., measured by the temperature monitor 120); information about a current orientation of the ablation probe 100 relative to anatomical tissue, such as information about the direction of greatest heat propagation from the ablation probe 100; combinations thereof; and the like.

The coolant system 224 may control the cooling of the ablation probe 100, and includes a coolant reservoir 228. The coolant reservoir 228 may have one or more containers that house one or more coolants (e.g., water, saline, CO₂ gas, etc.). One or more pumps may be controlled to pump the coolant into and out of the ablation probe 100. In some embodiments, each inflow channel and each outflow channel of the ablation probe 100 may have a separate pump to adjust the flow rate of coolant flowing through each of the inflow channels and the outflow channels. The coolant may be fluidically communicated to the ablation probe 100 through one or more fluid conduits (e.g., through the inflow channel 124). In some embodiments, the coolant system 224 may comprise a suction mechanism that can be turned on (e.g., begin generating a vacuum or other negative pressure to remove coolant from the ablation probe 100) when coolant is supplied to the ablation probe 100. As such, coolant may be dispensed into a distal end 106 of the ablation probe 100, such as by the inflow channel 124, and may be removed from the distal end 106 by the outflow channel 128. The coolant may then be pumped back into a separate container in the coolant reservoir 228. In some embodiments, the coolant system 224 may be controlled by the device 202 and/or by input commands by the user (e.g., via the user interface 210).

The database 230 may store information related to one or more surgical plans (e.g., information related to the type of tissue to be ablated, the position and orientation of one or more anatomical elements of a patient, etc.); information related to the ablation probe 100 (e.g., a model type, a recommended operating temperature or power range, etc.); and/or any other useful information. The database 230 may be configured to provide any such information to the device 202 or to any other device of the system 200 (e.g., to the generator 212, to the display 220, to the coolant system 224) or external to the system 200, whether directly or via the cloud 234. In some embodiments, the database 230 may be or comprise part of a hospital image storage system, such as a picture archiving and communication system (PACS), a health information system (HIS), and/or another system for collecting, storing, managing, and/or transmitting electronic medical records or other medical information.

The cloud 234 may be or represent the Internet or any other wide area network. The device 202 may be connected to the cloud 234 via the communication interface 208, using a wired connection, a wireless connection, or both. In some embodiments, the device 202 may communicate with the database 230 and/or an external device via the cloud 234.

The system 200 or similar systems may be used, for example, to carry out one or more aspects of any of the methods described herein. The system 200 or similar systems may also be used for other purposes.

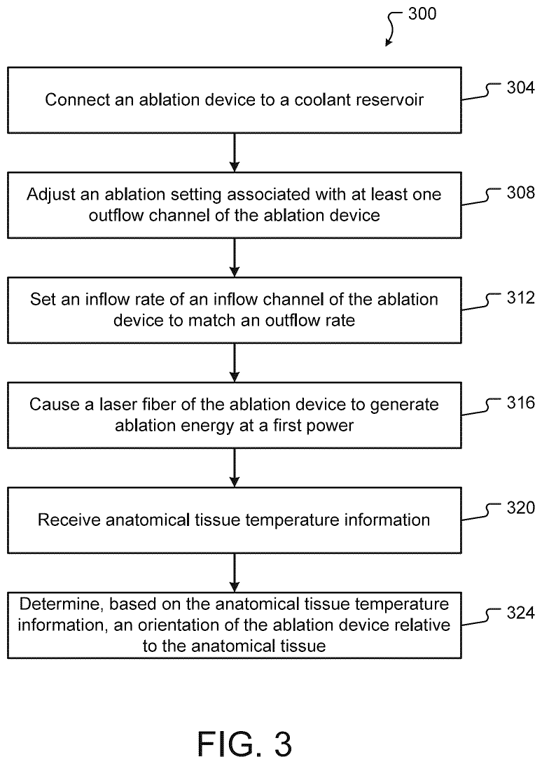

FIG. 3 depicts a method 300 that may be used, for example, to determine an orientation of an ablation device relative to anatomical tissue, such as when the ablation device has been inserted into the anatomical tissue during the course of a surgery or surgical procedure.

The method 300 (and/or one or more steps thereof) may be carried out or otherwise performed, for example, by at least one processor. The at least one processor may be the same as or similar to the processor(s) 204 of the device 202 described above. A processor other than any processor described herein may also be used to execute the method 300. The at least one processor may perform the method 300 by executing elements stored in a memory such as the memory 206. The elements stored in memory and executed by the processor may cause the processor to execute one or more steps of a function as shown in method 300. One or more portions of the method 300 may be performed by the processor executing any of the contents of the memory 206.

The method 300 comprises connecting an ablation device to a coolant reservoir (step 304). The ablation device may be similar to or the same as the ablation probe 100, and the coolant reservoir may be similar to or the same as the coolant reservoir 228. The inflow channel 124 and the outflow channel 128 are connected to the coolant reservoir 228, such that coolant from the coolant reservoir 228 can flow into the ablation probe 100 through the inflow channel 124 and then out of the ablation probe 100 and into the coolant reservoir 228. In some embodiments, a first container of the coolant reservoir 228 may provide the coolant flowing into the ablation probe 100, while a second, different container of the coolant reservoir 228 may receive the spent cooling flowing out of the ablation probe 100. In some embodiments, the step 304 may occur after the ablation probe 100 has been inserted into the target surgical site to ablate anatomical tissue (e.g., brain tissue).

The method 300 also comprises adjusting an ablation setting associated with at least one outflow channel of the ablation device (step 308). The at least one outflow channel may be any outflow channel described herein (e.g., the outflow channel 128) or any similar outflow channel. The ablation setting may be or comprise a change in an outflow rate through the outflow channel. For example, the suction rate associated with the outflow channel may be increased, and/or the pump connected to the outflow channel may generate a greater negative pressure, to increase the coolant outflow rate through the outflow channel. In some embodiments, a differential suction may be implemented, where a first outflow channel is set to a higher outflow rate than a second outflow channel, such that a temperature gradient is created. The temperature gradient may exist between the two outflow channels, between anatomical tissue proximate each of the two outflow channels, combinations thereof, and the like. In some embodiments, the outflow rates of one or more outflow channels in one or more sections (e.g., sections 138A-138C) may be adjusted to establish the temperature gradient when the laser fiber 116 generates ablation energy.

The ablation setting may be adjusted automatically by the processor 204 or other processor based on, for example, information about a surgical plan obtained from the database 230. Additionally or alternatively, the user may provide input through the user interface 210 to choose or adjust the outflow rate of the coolant through the outflow channel.

The method 300 also comprises setting an inflow rate of the ablation device to match an outflow rate (step 312). To ensure that the total amount of coolant in the ablation probe 100 remains stable during the course of the surgery or surgical procedure, the inflow rate of coolant may be set to match the overall outflow rate of coolant. For example, once the ablation settings have been determined (e.g., based on user input, based on information from the database 230, etc.), the processor 204 may determine the required inflow rate of coolant, and instruct the coolant system 224 to pump coolant from the coolant reservoir 228 into the ablation probe 100 at the specified inflow rate such that the amount of coolant present in the ablation probe 100 remains approximately the same over the course of the surgery or surgical procedure.

The method 300 also comprises causing a laser fiber of the ablation device to generate ablation energy at a first power (step 316). Once the coolant is flowing through the ablation probe 100, the laser fiber 116 may be caused to generate ablation energy. In some embodiments, the processor 204 may instruct the generator 212 to generate current that passes into laser fiber 116 to generate the ablation energy. The ablation energy may be generated at a power falling below a threshold power required for ablation. In other words, the laser fiber may generate sufficient heat to heat the surrounding anatomical tissue, but not enough heat to ablate the anatomical tissue.

The method 300 also comprises receiving anatomical tissue temperature information (step 320). The anatomical tissue temperature information may be obtained by capturing one or more thermal images of the anatomical tissue after the ablation probe 100 has heated the anatomical tissue. The thermal images may comprise information associated with the heating of the anatomical tissue, such as the distribution of heat dissipated from the ablation probe 100 and absorbed by the anatomical tissue. The images may be captured by a thermographic camera (e.g., an infrared camera). In some embodiments, MR Thermography may be used to capture temperature changes around the ablation probe 100. The MR Thermography may include capturing information about proton resonance frequency shift of the anatomical tissue surrounding the ablation probe 100 (due to the heat generated by the ablation probe 100 and received by the anatomical tissue), and using such information to generate a temperature map. In some embodiments, the anatomical tissue temperature information (e.g., the temperature of the anatomical tissue, the thermal images depicting the anatomical tissue, combinations thereof, and/or the like) may be rendered to the display 220 to enable the physician to view the overall distribution of heat in the anatomical tissue.

Additionally or alternatively, one or more temperature sensors may be used to capture the anatomical tissue temperature information. The one or more temperature sensors may be similar to the temperature monitor 120, and may be disposed proximate patient tissue to monitor the temperature thereof. Based on readings from the one or more temperature sensors, the processor 204 may determine the temperature of the anatomical tissue. In some embodiments, the processor 204 may render such readings and determined temperatures to the display 220.

The method 300 also comprises determining, based on the anatomical tissue temperature information, an orientation of the ablation device relative to the anatomical tissue (step 324). The anatomical tissue temperature information may indicate the direction in which heat is being dissipated from the ablation probe 100. The direction may be defined in as posterior direction (e.g., a direction pointing toward the back of the patient), an anterior direction (e.g., a direction pointing to the front of the patient), a medial direction (e.g., a direction pointing to the midline of the patient), a lateral direction (e.g., a direction pointing away from the midline of the patient), or a combination thereof such as an anterior-medial direction, an anterior-lateral direction, a posterior-lateral direction, or a posterior-medial direction. In some embodiments, the direction may be or comprise the direction of greatest heat dissipation from the ablation probe 100.

The processor 204 may use the direction to determine or define the orientation of the ablation probe 100 relative to the anatomical tissue. In some embodiments, the direction may be defined based on an angle of the direction relative to a known coordinate system (e.g., the coordinate system 102). For example, the processor 204 may define the direction has having an angle of 0 degrees when the greatest direction of heat dissipation points along the X-axis of the ablation probe 100, an angle of 90 degrees when the greatest direction of heat dissipation points along the Y-axis direction of the ablation probe 100, an angle of 180 degrees when the greatest direction of heat dissipation points along the negative X-axis direction, etc. As a result, the orientation of the ablation probe 100 relative to the anatomical tissue may comprise an angle reflecting the direction of the greatest heat dissipation.

The processor 204 may also use information about the various ablation settings of the ablation probe 100, such as the ratio of outflow rates between different outflow channels (e.g., the ratio of the outflow rate of the first outflow channel 144A to the outflow rate of the second outflow channel 144B), to determine or define the orientation of the ablation probe 100. For example, when the ratio between an outflow rate of a first outflow channel and an outflow rate of a second outflow channel is a first value (e.g., 0.5, indicating that the outflow rate of the second outflow channel is double the outflow rate of the first outflow channel), the direction of highest heat dissipation is at 0 degrees, and the ablation probe 100 may be defined as being in a first orientation relative to the anatomical tissue. When the ratio is a second value (e.g., 2, indicating that the outflow rate of the first outflow channel is double the outflow rate of the second outflow channel) and the direction of highest temperature increase of the anatomical tissue is at 180 degrees, the ablation probe 100 may be defined as being in a second orientation relative to the anatomical tissue. In some embodiments, processor 204 may repeat the steps 308, 312, 316, and 320 with various ablation settings on the ablation probe 100 to determine a spectrum of ratios and directions, such that the primary direction of heat dissipation can be effectively steered by choosing the appropriate ratio of outflow rates.

In some embodiments, the orientation of the ablation probe 100 relative to the anatomical tissue may be defined for the entirety of the surgery or surgical procedure, while in other embodiments the orientation may be changed by the physician during the surgery or surgical procedure, such as when the physician desires to recalibrate the ablation probe 100 by repeating the performance of the steps 308-324.

Based on the determined orientation, the ratios of the outflow rates of the outflow channels may be adjusted (e.g., by the processor 204, by a physician, etc.) to change the direction of heat dissipated from the ablation probe 100 and into the anatomical tissue, as discussed below. The change in direction by adjustment of outflow rates of coolant may enable ablation of target anatomical tissue without needing to adjust the position of the ablation probe 100 and/or components thereof (e.g., laser fiber 116).

It is to be understood that, while the ratio of outflow rates is discussed above, additional or alternative ratios may be used to adjust the direction of heat dissipation of the ablation probe 100. Some non-limiting examples of ratios that can be associated with and adjusted to change the direction of heat dissipation of the ablation probe 100 include a ratio of inflow rate of a first coolant type to an inflow rate of a second coolant type; a ratio in the temperature of a first coolant to the temperature of a second coolant; a ratio of a number of inflow channels to a number of used outflow channels (or vice versa); and the like.

The present disclosure encompasses embodiments of the method 300 that comprise more or fewer steps than those described above, and/or one or more steps that are different than the steps described above.

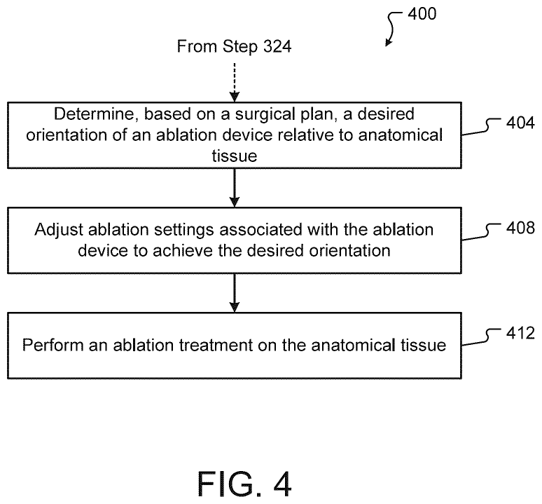

FIG. 4 depicts a method 400 that may be used, for example, to ablate anatomical tissue with an ablation device. In some embodiments, the method 400 may continue from the step 324 of the method 300.

The method 400 (and/or one or more steps thereof) may be carried out or otherwise performed, for example, by at least one processor. The at least one processor may be the same as or similar to the processor(s) 204 of the device 202 described above. A processor other than any processor described herein may also be used to execute the method 400. The at least one processor may perform the method 400 by executing elements stored in a memory such as the memory 206. The elements stored in memory and executed by the processor may cause the processor to execute one or more steps of a function as shown in method 400. One or more portions of the method 400 may be performed by the processor executing any of the contents of the memory 206.

The method 400 comprises determining, based on a surgical plan, a desired orientation of an ablation device relative to anatomical tissue (step 404). In some embodiments, the step 404 may continue from the step 324 of the method 300, where the orientation of the ablation probe 100 relative to the anatomical tissue was determined and/or defined. The processor 204 may access the database 230 and access the surgical plan to determine the pose of the target anatomical tissue relative to the pose of the ablation probe 100. Based on the surgical plan, the processor 204 may determine the desired direction of heat dissipation from the ablation probe 100 to ablate the target anatomical tissue.

The method 400 also comprises adjusting the ablation settings associated with the ablation device to achieve the desired orientation (step 408). The ablation settings of the ablation probe 100 may be adjusted by the processor 204 and/or the user based on the desired orientation of the ablation probe 100. For example, the inflow rates and/or outflow rates of the coolant, the type and/or temperature of the coolant, and or the like may be adjusted to change the heat map associated with the ablation probe 100. In other words, the ablation probe 100 may be adjusted such that the orientation of heat dissipated by the ablation probe 100 changes to the desired orientation.

In some embodiments, the processor 204 may determine a required ratio (e.g., an outflow ratio of the outflow rate of a first outflow channel to the outflow rate of a second outflow channel) to effectively steer the direction of the heat dissipation to the desired orientation. For example, a range of various ratios and the corresponding direction of heat dissipation may be saved in the database 230, and the processor 204 may access the range to determine the required ratio to change the direction of the heat dissipation. The processor 204 may then set the outflow rates and/or other ablation settings in accordance with the ratio to change the direction of the heat dissipation.

To confirm the ablation settings, the step 408 may comprise performing any one or more of the steps 312, 316, 320, and 324 of the method 300. For instance, once the outflow rate of one or more outflow channels is set, the laser fiber 116 may be caused to generate ablation energy at a sub-ablation threshold power. Then, based on one or more temperature sensors and/or thermal images, the heat profile of the anatomical tissue (e.g., the direction of highest heat dissipation) may be determined. The determined direction of the heat dissipation may then be compared to the orientation determined in the step 324, to confirm the direction of heat dissipation.

The method 400 also comprises performing an ablation treatment on the anatomical tissue (step 412). The processor 204 may cause the generator 212 to generate current at or above an ablation threshold, such that the ablation probe 100 generates sufficient heat to cause ablation of the anatomical tissue in the orientation direction determined and confirmed in the step 408. In some embodiments, the ablation of the anatomical tissue may be confirmed by capturing one or more images (e.g., Computed Tomography (CT) scans, Magnetic Resonance Imaging (MRI) scans, etc.) of the anatomical tissue. In some embodiments, the method 400 or one or more steps thereof may be repeated for other anatomical tissues proximate the ablation probe 100.

The present disclosure encompasses embodiments of the method 400 that comprise more or fewer steps than those described above, and/or one or more steps that are different than the steps described above.

As noted above, the present disclosure encompasses methods with fewer than all of the steps identified in FIGS. 3 and 4 (and the corresponding description of the methods 300 and 400), as well as methods that include additional steps beyond those identified in FIGS. 3 and 4 (and the corresponding description of the methods 300 and 400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system, comprising:
an ablation device, comprising:
a housing extending from a proximal end to a distal end;
a laser fiber disposed at least partially within the housing, wherein the laser fiber is configured to generate ablation energy dissipated from the distal end of the housing to ablate anatomical tissue;
at least one inflow channel disposed at least partially within the housing, the at least one inflow channel fluidically connectable to a coolant reservoir to dispense a coolant into the distal end of the housing; and
a plurality of outflow channels each disposed at least partially within the housing and that enable removal of the coolant from the distal end of the housing, wherein the at least one inflow channel and the plurality of outflow channels are positionable in different sections of the housing to control a heat profile associated with the ablation energy;
a processor; and
a memory storing data thereon that, when executed by the processor, enable the processor to:
determine a desired orientation of the ablation device relative to the anatomical tissue;
adjust an ablation setting associated with the ablation device to achieve the desired orientation; and
cause the laser fiber to generate the ablation energy.

2. The system of claim 1, wherein the at least one inflow channel comprises two inflow channels.

3. The system of claim 1, wherein the plurality of outflow channels comprises three outflow channels.

4. The system of claim 1, wherein the ablation setting comprises a temperature of the coolant, an inflow rate of the coolant, an outflow rate of the coolant, a type of coolant, or a combination thereof.

5. The system of claim 4, wherein the data, when processed by the processor, further enable the processor to:
change the type of coolant from a first coolant type to a second coolant type.

6. The system of claim 1, wherein the plurality of outflow channels comprises a first outflow channel and a second outflow channel, wherein the first outflow channel has a first outflow rate, and wherein the second outflow channel has a second outflow rate different than the first outflow rate.

7. The system of claim 1, wherein the processor causes the ablation energy to be generated at a first power at a first time, and at a second power greater than the first power at a second time later than the first time.

8. The system of claim 7, wherein the data, when processed by the processor, further enable the processor to:
receive, when the ablation energy is generated at the first power, anatomical tissue temperature information associated with a temperature of the anatomical tissue; and
adjust, based on the anatomical tissue temperature information and a surgical plan, the ablation setting associated with the ablation device.

9. The system of claim 1, wherein the different sections comprise a first section and a second section, wherein the at least one inflow channel is disposed in the first section, and wherein at least one outflow channel of the plurality of outflow channels is disposed in the second section.

10. The system of claim 1, wherein the different sections comprise a first section, a second section, and a third section, wherein the laser fiber and the at least one inflow channel are disposed in the first section, wherein a first outflow channel of the plurality of outflow channels is disposed in a second section, and wherein a second outflow channel of the plurality of outflow channels is disposed in the third section.

11. The system of claim 1, wherein the different sections comprise a first section, a second section, a third section, and a fourth section, wherein the laser fiber and the at least one inflow channel are disposed in the first section, wherein a first outflow channel of the plurality of outflow channels is disposed in the second section, wherein a second outflow channel of the plurality of outflow channels is disposed in the third section, and wherein a third outflow channel of the plurality of outflow channels is disposed in the fourth section.

12. A method, comprising:
determining a current orientation of an ablation device relative to anatomical tissue;
determining a desired orientation of the ablation device relative to the anatomical tissue;
adjusting an ablation setting associated with the ablation device to control a heat profile of ablation energy associated with the ablation device to achieve the desired orientation; and
causing a laser fiber of the ablation device to generate the ablation energy.

13. The method of claim 12, further comprising:
connecting the ablation device to a coolant reservoir; and
setting an inflow rate of a coolant into the ablation device to match an outflow rate of the coolant from the ablation device.

14. The method of claim 12, wherein adjusting the ablation setting comprises:
changing an outflow rate of at least one outflow channel of a plurality of outflow channels associated with the ablation device.

15. The method of claim 12, wherein determining the current orientation of the ablation device comprises:
causing the laser fiber to generate the ablation energy at a first power; and
receiving anatomical tissue temperature information associated with a temperature of the anatomical tissue.

16. The method of claim 12, further comprising:
adjusting, based on the anatomical tissue temperature information and a surgical plan, the ablation setting associated with the ablation device.

17. The method of claim 16, wherein the ablation energy at the first power is insufficient to ablate the anatomical tissue.

18. The method of claim 12, wherein the ablation setting comprises a temperature of a coolant, an inflow rate of the coolant, an outflow rate of the coolant, a type of coolant, or a combination thereof.

19. The method of claim 18, further comprising:
changing the type of coolant from a first coolant type to a second coolant type.
